(19) United States
(12) Patent Application Publication
(10) Pub. No.: US 2009/0252711 A1
(43) Pub. Date: Oct. 8, 2009

(54) Stem Cells And Methods Of Making And Using Stem Cells

(76) Inventors: Andrew Craig Boquest, Victoria (AU); Phillipe Collas, Oslo (NO)

Correspondence Address: Casimir Jones, S.C., 440 Science Drive, Suite 203, Madison, WI, 53711, US

(21) Appl. No.: 12/300,422

(22) PCT Filed: May 11, 2007
(86) PCT No.: PCT/IB07/03767

§ 371 Date: Jun. 15, 2009

Related U.S. Application Data
(60) Provisional application No. 60/799,339, filed on May 11, 2006.

(51) Int. Cl. A61K 35/12; C12N 5/06; C12Q 1/68; A01K 67/027

(52) U.S. Cl. 424/93.7; 435/377; 435/354; 435/351; 435/350; 435/366; 435/363; 435/6; 800/25

(57) ABSTRACT

The invention provides a method of making a pluripotent stem cell from a cell that is not pluripotent, such as from a differentiated stem cell or a lineage-restricted stem cell. The methods comprise culturing the starting cell in the presence of one or more epigenetic altering agents, such as a histone deacetylase inhibitor and/or a DNA methyltransferase inhibitor. Pluripotent stem cells are also provided, as are methods of treating or preventing a disease, disorder, or condition in a mammal using the cells.

The present application claims priority to the U.S. Provisional Application No. 60/799,339 filed May 11, 2006 and PCT Patent Application Number PCT/IB2007/003767 filed May 11, 2007.

### FIELD OF THE INVENTION

The invention provides a method of making a pluripotent stem cell from a cell that is not pluripotent, such as from a differentiated stem cell or a lineage-restricted stem cell. The methods comprise culturing the starting cell in the presence of one or more epigenetic altering agents, such as a histone deacetylase inhibitor and/or a DNA methyltransferase inhibitor. Pluripotent stem cells are also provided, as are methods of treating or preventing a disease, disorder, or condition in a mammal using the cells.

### BACKGROUND OF THE INVENTION

The cells of a mammal all contain essentially the same genome, yet a mammal contains diverse cell types. This diversity is determined by the repertoire of genes expressed in each cell. Differences in gene expression are mediated in part by regulation of transcription on a gene by gene basis. Those differences are also mediated by epigenetic mechanisms, which include differences in DNA methylation, as well as differences in chromatin structure involving histone modifications.

Epigenetic inheritance systems (EISs) allow cells of different phenotype but identical genotype to transmit their phenotype to their offspring, even when the phenotype-inducing stimuli are absent, as is often the case. One basis of epigenetic inheritance is mediated by proteins or chemical groups that are attached to DNA and modify its activity, by acting as chromatin marks. These marks are copied with the DNA. For example, several cytosines in eukaryotic DNA are methylated (5-methylcytosine). The number and pattern of such methylated cytosines influences the functional state of the gene: low levels of methylation correspond to high potential activity while high levels correspond to low activity. While there are random changes in the methylation pattern, there are also very specific ones, induced by environmental factors. After DNA replication, maintenance DNA methyltransferases make sure the methylation pattern of the parental DNA is copied to the daughter strand.

Histone acetylation is another epigenetic mechanism. One way the expression of a gene can be enhanced is through the acetylation on lysines of the N-terminus tails of the internal histones of the nucleosome. Since lysine normally has a positive charge on the nitrogen at its end, it can bind the negatively charged phosphates of the DNA backbone and prevent them from repelling each other. When the charge is neutralized, the DNA can fold tightly, thus preventing access to the DNA by the transcriptional machinery, and keeping transcription low or off in the surrounding chromatin. When an acetyl group is added to the +NH₂ of the lysine, it removes the positive charge and causes the DNA to repel itself and not fold up so tightly. When this occurs, complexes like SWI/SNF and other transcriptional factors can bind to the DNA, thus opening it up and exposing it to enzymes like RNA polymerase so transcription of the gene can occur.

During development of a mammalian embryo the initially totipotent cells of the blastula become progressively more restricted in developmental potential. One mechanism that appears to drive that process of restriction is epigenetic factors that fix the transcriptional status of regions of the genome. Thus, for example, as cells of the embryo are partitioned to mesodermal cell fates epigenetic changes to chromatin may silence regions of the genome that include genes unique to ectodermal cell fates, such as genes unique to the central nervous system.

A role for epigenetic altering agents in cell differentiation has been described. For example, Cho et al., J. of Cellular Biochemistry, Vol. 96, pp. 533-542 (2005) report that treatment of cultured ASCs with histone deacetylase inhibitors trichostatin A and Valproic acid leads to enhanced differentiation towards the osteogenic (bone) lineage. Jori, F. P., et al., Cell Death Differ., Vol. 12(1): 65-77 (2005), report that the role of RB2/p130 and RB genes in differentiation of bone marrow mesenchymal stem cells appear to rely, at least in part, on the activity of the histone deacetylase inhibitor trichostatin A. Araki, H. et al., Exp Hematol., Vol. 34(2): 140-9 (2006), report that the combination treatment of 5-aza-2′-deoxycytidine, a DNA methyltransferase inhibitor, and trichostatin A, a histone deacetylase inhibitor, resulted in enhanced proliferation of hematopoietic stem cells in vitro. The authors conclude that the treatment results in enhanced self renewal of HSCs. Schmittwolf, C., et al., EMBO J., Vol. 24(3): 554-66 (2005), report that the treatment of neurosphere stem cells with 5-aza-2′-deoxycytidine, a DNA methyltransferase inhibitor, and trichostatin A, a histone deacetylase inhibitor in combination resulted in haematopoietic repopulation in irradiated mice. Milhem, M., et al., Blood, Vol. 103(11): 4102-10 (2004), report that the combination treatment of 5-aza-2′-deoxycytidine, a DNA methyltransferase inhibitor, and trichostatin A, a histone deacetylase inhibitor, resulted in enhanced proliferation of hematopoietic stem cells in vitro and that expanded cells could rescue the blood forming ability in lethally irradiated mice. Marin-Husstege, M., et al., J Neurosci., Vol. 22(23): 10333-45 (2002), report that trichostatin A blocked the differentiation of neural stem cells towards the oligodendrocyte cell type, but not towards the astrocyte cell type. Mukhopadhyay, N. K. et al., J Cell Mol Med., Vol. 9(3): 662-9 (2005), describe that telomerase activity was induced in lung fibroblast cells as a result of trichostatin A treatment. Such cells were not subsequently tested for their ability to differentiate into tissues of the 3 germ layers. Hou, M., et al., Exp Cell Res., Vol. 274(1): 25-34 (2005), describe the upregulation of the gene hTERT, the catalytic component of telomerase in normal human cells when exposed to trichostatin A.

This invention is based, in part, on the inventors' discovery that epigenetic altering agents, such as histone deacetylase inhibitors and DNA methyltransferase inhibitors may be used to reverse or neutralize the epigenetic coding in cells, including in lineage-restricted stem cells. Among other things, this invention provided methods of using epigenitic altering agents to make a pluripotent stem cell from a non-pluripotent stem cell, such as a lineage-restricted stem cell. These cells find many uses, such as in the therapeutic prevention and/or treatment of diseases, disorders, or conditions.

### SUMMARY OF THE INVENTION

The invention provides a method of making a pluripotent stem cell from a cell that is not pluripotent, such as from a differentiated stem cell or a lineage-restricted stem cell. The methods comprise culturing the starting cell in the presence of one or more epigenetic altering agents, such as a histone deacetylase inhibitor and/or a DNA methyltransferase inhibitor. Pluripotent stem cells are also provided, as are methods of treating or preventing a disease, disorder, or condition in a mammal using the cells.

Certain illustrative embodiments of the invention are described below. The present invention is not limited to these embodiments.

In an embodiment the invention provides a method of making a pluripotent stem cell from a cell that is not pluripotent. In an embodiment, the method comprises isolating a non-pluripotent cell from a tissue of a mammal; and culturing the non-pluripotent cell in the presence of one or more epigenetic altering agents, under conditions that allow formation of a pluripotent stem cell, to thereby provide the pluripotent stem cell. In an embodiment of the method the isolated non-pluripotent cell is in a G₀ state. In another embodiment of the method the isolated non-pluripotent cell is terminally differentiated.

In a further embodiment the invention provides a method of making a pluripotent stem cell from a lineage-restricted stem cell. In an embodiment, the method comprises isolating a lineage-restricted stem cell from a tissue of a mammal; and culturing the lineage-restricted stem cell in the presence of one or more epigenetic altering agents, under conditions that allow formation of a pluripotent stem cell, to thereby provide the pluripotent stem cell. In an embodiment of the method the isolated lineage-restricted stem cell is in a G₀ state. In other embodiments of the method the lineage-restricted stem cell is a somatic stem cell, which may be adipose tissue-derived, or a stromal stem cell, or a CD45−, CD34+, CD105+ cell, which may also be CD31−. In another embodiment of the method the lineage-restricted stem cell is obtained from a tissue selected from adipose, bone marrow, blood, brain, muscle, skin, liver, and pancreas. In another embodiment of the method the lineage-restricted stem cell is selected from an adipose tissue stromal stem cell, bone marrow stromal stem cell, hematopoietic stem cell, hair follicle stem cell, neural stem cell, muscle stem cell, cord blood stem cell, skin stem cell, liver stem cell, and pancreatic stem cell. In other embodiments of the method the one or more epigenetic altering agents may comprise a histone deacetylase inhibitor, which may be trichostatin A, or a DNA methyltransferase inhibitor, which may be 5-azacytidine, or both a histone deacetylase inhibitor and a DNA methyltransferase inhibitor, which may be trichostatin A and 5-azacytidine.

In another embodiment of the method one or more chromatin marker of an increased developmental potential is increased in the pluripotent stem cell relative to the lineage-restricted stem cell following isolation but before culture in the presence of the epigenetic altering agent. In another embodiment of the method the chromatin modification comprises a reduction in DNA methylation, while in another embodiment the chromatin modification comprises an increase in histone acetylation, while in another embodiment of the method the chromatin modification comprises a reduction in DNA methylation and an increase in histone acetylation.

In another embodiment of the method the pluripotent stem cell expresses one or more mRNA or protein pluripotent stem cell marker that was not expressed in the lineage-restricted stem cell following isolation but before culture in the presence of the epigenetic altering agent. In another embodiment of the method the pluripotent stem cell expresses 1, 1-5, 5-10, 10-20, 20-50, 50-100, 100-200, 200-1000, or over 1000 mRNA or protein pluripotent stem cell markers that were not expressed in the lineage-restricted stem cell following isolation but before treatment with the epigenetic altering agent. In another embodiment of the method the one or more mRNA or protein marker is one or more of OCT-4, TERT, NANOG, SSEA-1, TRA-1-60, TRA-1-81, AC133, CD9, FLT3, C-KIT, REX-1, STELLA, SOX-2, UTF1, OXT2, LEFTY-1, FOXD3, DNMT3A, DNMT3B, and FGF2.

In another embodiment of the method the mammal is a human, cow, sheep, big-horn sheep, goat, buffalo, antelope, oxen, horse, donkey, mule, deer, elk, caribou, water buffalo, camel, llama, alpaca, rabbit, pig, mouse, rat, guinea pig, hamster, dog, cat, or primate, such as a monkey.

In another embodiment of the method the isolated lineage-restricted stem cell is a progeny of at least one cell division that occurred in vitro, prior to culturing in the presence of one or more epigenetic altering agents.

In a further embodiment the invention provides a pluripotent stem cell made by a method comprising isolating a lineage-restricted stem cell from a tissue of a mammal; and culturing the lineage-restricted stem cell in the presence of one or more epigenetic altering agents, under conditions that allow formation of a pluripotent stem cell, to thereby provide the pluripotent stem cell.

In a further embodiment the invention provides a method for making a pluripotent stem cell from a lineage-restricted stem cell. In an embodiment, the method comprises isolating a lineage-restricted stem cell from a tissue of a mammal, wherein the lineage-restricted stem cell expresses one or more mRNA or protein markers that define its lineage restriction; and exposing the lineage-restricted stem cell to one or more epigenetic altering agents, to thereby provide the pluripotent stem cell; wherein the pluripotent stem cell expresses one or more mRNA or protein pluripotent stem cell marker that was not expressed in the lineage-restricted stem cell following isolation but before treatment with the epigenetic altering agent; and wherein the pluripotent stem cell does not express one or more mRNA or protein marker that define the lineage restriction of the lineage-restricted stem cell from which it was made. In an embodiment of the method the pluripotent stem cell is a progeny of the lineage-restricted stem cell.

In a further embodiment the invention provides a method of providing a reprogrammed cell. In an embodiment, the method comprises making a pluripotent stem cell from a lineage-restricted stem cell, by a method comprising isolating a lineage-restricted stem cell from a tissue of a mammal; and culturing the lineage-restricted stem cell in the presence of one or more epigenetic altering agents, under conditions that allow formation of a pluripotent stem cell, to thereby provide the pluripotent stem cell; and culturing the pluripotent stem cell in one or more reprogramming agents, under conditions that allow formation of a reprogrammed cell, to thereby provide the reprogrammed cell. In an embodiment of the method the reprogrammed cell expresses one or more mRNA or protein reprogramming marker that was not expressed in the lineage-restricted stem cell. In another embodiment of the method the reprogrammed cell is a lineage-restricted stem cell. In another embodiment, the invention provides a reprogrammed cell made by the method.

In a further embodiment the invention provides a method of treating or preventing a disease, disorder, or condition in a mammal. In an embodiment the method comprises isolating a lineage-restricted stem cell from a tissue of a mammal; culturing the lineage-restricted stem cell in the presence of one or more epigenetic altering agents, under conditions that allow formation of a pluripotent stem cell, to thereby provide the pluripotent stem cell; and administering the pluripotent stem cell to a mammal in need of cells derived from the pluripotent stem cell. In another embodiment of the method the lineage-restricted stem cell is isolated from the mammal that receives the pluripotent stem cell.

In a further embodiment the invention provides a method of treating or preventing a disease, disorder, or condition in a mammal. In an embodiment, the method comprises making a pluripotent stem cell from a lineage-restricted stem cell, by a method comprising isolating a lineage-restricted stem cell from a tissue of a mammal; and culturing the lineage-restricted stem cell in the presence of one or more epigenetic altering agents, under conditions that allow formation of a pluripotent stem cell, to thereby provide the pluripotent stem cell; culturing the pluripotent stem cell in one or more reprogramming agents, under conditions that allow formation of a reprogrammed cell, to thereby provide the reprogrammed cell; and administering the reprogrammed cell to a mammal in need of cells of the reprogrammed cell type. In an embodiment of the method the lineage-restricted stem cell is isolated from the mammal that receives the reprogrammed cell. In another embodiment, the invention provides a mammal comprising a cell administered by the method of claim 39.

In a further embodiment the invention provides a method of inducing or increasing expression of one or more mRNA or protein pluripotent stem cell markers in a cell. In an embodiment, the method comprises determining the expression level of the one or more mRNA or protein pluripotent stem cell markers in the cell when the cell is cultured in the absence of one or more epigenetic altering agents; culturing the cell in the presence of the one or more epigenetic altering agents; determining the expression level of the one or more mRNA or protein pluripotent stem cell markers in the cell when the cell is cultured in the presence of the one or more epigenetic altering agents; and comparing the expression level of the one or more mRNA or protein pluripotent stem cell markers when the cell is cultured in the presence of the one or more epigenetic altering agents, with the expression level of the one or more mRNA or protein pluripotent stem cell markers when the cell is cultured in the absence of the one or more epigenetic altering agents, and determining that the expression level of the one or more mRNA or protein pluripotent stem cell markers is induced or increased when the cell is cultured in the presence of the one or more epigenetic altering agents. In an embodiment of the method the one or more epigenetic altering agents is a histone deacetylase inhibitor, which may be trichostatin A.

In another embodiment of the method the one or more epigenetic altering agents is a DNA methyltransferase inhibitor, which may be 5-azacytidine. In another embodiment the one or more epigenetic altering agents comprises a histone deacetylase inhibitor and a DNA methyltransferase inhibitor, which may be trichostatin A and 5-azacytidine.

In another embodiment of the method the expression level of 2-5, 5-10, 10-20, 20-50, 50-100, 100-200, 200-1000, or over 1000 mRNA or protein pluripotent stem cell markers is induced or increased by culturing the cell in the presence of the one or more epigenetic altering agents. In another embodiment of the method the one or more mRNA or protein marker is selected from OCT-4, TERT, NANOG, SSEA-1, SSEA-4, TRA-1-60, TRA-1-81, AC133, CD9, FLT3, C-KIT, REX-1, STELLA, SOX-2, UTF1, OXT2, LEFTY-1, FOXD3, DNMT3A, DNMT3B, and FGF2.

In another embodiment of the method the cell is a lineage-restricted stem cell, which may be a somatic stem cell, which may be adipose tissue-derived. In another embodiment of the method the lineage-restricted stem cell is a stromal stem cell. In another embodiment of the method, the lineage-restricted stem cell is a CD45−, CD34+, CD 105+ cell, which may also be CD31−.

In a further embodiment the invention provides a method of making a cloned mammalian cell. In an embodiment, the method comprises isolating a non-pluripotent cell from a tissue of a mammal; culturing the cell in the presence of one or more epigenetic altering agents, under conditions that allow the chromatin of the cell to be modified such that the developmental potential of the cell is increased; transferring the nucleus of the cell with an increased developmental potential into an enucleated recipient cell; and allowing the recipient cell to undergo one or more cell divisions to provide the cloned mammalian cell. In another embodiment of the method the cloned mammalian cell is a pluripotent stem cell. In another embodiment, the method further comprises culturing the pluripotent stem cell in one or more reprogramming agents, under conditions that allow formation of a reprogrammed cell, to thereby provide a reprogrammed cell. In another embodiment, the reprogrammed cell is a lineage-restricted stem cell.

In another embodiment, the invention a method of treating or preventing a disease, disorder, or condition in a mammal. The method comprises making a cloned mammalian cell as described in the preceding paragraph; and administering the cloned cell to a mammal in need thereof.

In a further embodiment, the invention provides a method of making a cloned mammal. The method comprises making a cloned cell as described above and transferring the cell into a recipient blastula; transferring the blastula into a recipient mother; and allowing the blastula to develop to term to thereby provide the cloned mammal. In another embodiment, a method of making a cloned cell is provided, comprising isolating a cloned cell from the cloned mammal. In another embodiment, a method of treating or preventing a disease, disorder, or condition in a mammal, comprising isolating a cloned cell from the cloned mammal; and administering the cloned cell to a mammal in need thereof.

In a further embodiment, the invention provides a method of making a cloned mammalian embryo. The method comprises making a cloned cell as described above and transferring the cell into a recipient blastula; transferring the blastula into a recipient mother; and allowing the blastula to develop to an embryonic stage to thereby provide the cloned mammal. In another embodiment, a method of making a cloned cell is provided, comprising isolating a cloned cell from the cloned mammalian embryo. In another embodiment, a method of treating or preventing a disease, disorder, or condition in a mammal, comprising isolating a cloned cell from the cloned mammalian embryo; and administering the cloned cell to a mammal in need thereof.

### Definitions

As used herein, a “stem cell” is a cell with the developmental potential to produce a more specialized cell type and at the same time to replicate itself. A stem cell may divide to produce two daughters that are themselves stem cells or it may divide to produce a daughter that is a stem cell and a daughter that is a more specialized cell type.

As used herein, a “pluripotent stem cell” is a stem cell with the developmental potential to produce ectodermal cell types, mesodermal cell types, and endodermal cell types. An embryonic stem cell is a type of “totipotent stem cell”. That is, it is a cell that can give rise to every cell type in a mammal. A “totipotent stem cell” is a type of “pluripotent stem cell”.

As used herein, a “lineage-restricted stem cell” is a stem cell that can only give rise to cell types within one germ layer (i.e., to cell types within ectoderm or mesoderm or endoderm lineages). The lineage-restricted stem cell may have the potential to give rise to all cell types within the germ layer or it may only have the potential to give rise to a subset of cell types within the germ layer.

As used herein, an “epigenetic altering agent” is an agent that modifies chromatin structure, directly and/or indirectly, to modify the developmental potential of a stem cell.

As used herein, a “pluripotent stem cell marker” is an mRNA or protein that is present in a pluripotent stem cell but absent in a lineage-restricted stem cell.

As used herein, a “lineage-restricted stem cell marker” is a marker that is present in a lineage-restricted stem cell but absent in a pluripotent stem cell. A “lineage-restricted stem cell marker” may also be unique to a single type of lineage-restricted stem cell, or may be present is some types of lineage-restricted stem cells but not others. Alternatively, a lineage-restricted stem cell marker may be present in all stem cells that are not totipotent or pluripotent.

As used herein, a “reprogramming agent” is an agent, such as a protein or a gene, that when introduced to or into a “pluripotent stem cell” can change that “pluripotent stem cell” into a “lineage-restricted stem cell” or into a “precursor cell” or a “differentiated cell type.”

As used herein, a “reprogrammed cell” is a “lineage-restricted stem cell” or a “precursor cell” or a “differentiated cell type,” which was formed by exposure of a “pluripotent stem cell” to a “reprogramming agent.”

A “precursor cell” is a cell that can self-renew and also divide to give rise to a “differentiated cell type.”

A “differentiated cell” is a cell that has lost the ability to self-renew.

A “somatic stem cell” is a stem cell found in or isolated from a differentiated tissue, that can renew itself and give rise to at least one specialized cell type of the germ layer from which it originated.

Non-limiting examples of somatic stem cells include “hematopoietic stem cells,” “bone marrow stromal stem cells,” “neural stem cells,” “epithelial stem cells,” and “skin stem cells,” for example. “Hematopoietic stem cells” give rise to all the types of blood cells: red blood cells, B lymphocytes, T lymphocytes, natural killer cells, neutrophils, basophils, eosinophils, monocytes, macrophages, and platelets. “Bone marrow stromal stem cells” give rise to a variety of cell types: bone cells (osteocytes), cartilage cells (chondrocytes), fat cells (adipocytes), and other kinds of connective tissue cells such as those in tendons. “Neural stem cells” in the brain give rise to its three major cell types: nerve cells (neurons) and two categories of non-neuronal cells—astrocytes and oligodendrocytes. “Epithelial stem cells” in the lining of the digestive tract occur in deep crypts and give rise to several cell types: absorptive cells, goblet cells, Paneth cells, and enteroendocrine cells. “Skin stem cells” occur in the basal layer of the epidermis and at the base of hair follicles. The epidermal stem cells give rise to keratinocytes, which migrate to the surface of the skin and form a protective layer. The follicular stem cells can give rise to both the hair follicle and to the epidermis.

As used herein, the term “treat,” “treating” or “treatment” refers to the administration of therapy to an individual who already manifests at least one symptom of a disease, disorder, or condition, or who has previously manifested at least one symptom of a disease, disorder, or condition, and includes inhibiting the disease, disorder, or condition, arresting its development, and relieving the disease, disorder, or condition, for example, by causing regression, or restoring or repairing a lost, missing, or defective function or cell type, or by stimulating an inefficient process.

As used herein, the term “prevent,” “preventing” and “prevention” refers to the administration of therapy an individual who may ultimately manifest at least one symptom of a disease, disorder, or condition, but who has not yet done so, to reduce the chance that the individual will develop the symptom of the disease, disorder, or condition over a given period of time. Such a reduction may be reflected, for example, in a delayed onset of the at least one symptom of the disease, disorder, or condition in the patient.

Mammals include, for example, humans, cows, sheep, big-horn sheep, goats, buffalos, antelopes, oxen, horses, donkeys, mule, deer, elk, caribou, water buffalo, camels, llama, alpaca, rabbits, pigs, mice, rats, guinea pigs, hamsters, dogs, cats, and primates such as monkeys. As used herein “mammal” includes embryonic, juvenile, and adult mammals, unless the context clearly indicates otherwise.

### DESCRIPTION OF THE INVENTION

Many methods of the invention begin with a cell that has been isolated from a mammal and that is not pluripotent, and then make a pluripotent stem cell or a reprogrammed cell from that starting cell. The starting cell may be any cell type of a mammal that is not pluripotent, including, for example, a differentiated cell, a precursor cell, or a lineage-restricted stem cell. The starting cell may be used directly upon isolation from the mammal or it may first be expanded in culture for a defined period of time, such as 1-5 doublings, 5-10 doublings, 10-20 doublings, 20-50 doublings, 50-100 doublings, or more than 100 doublings; alternatively, the period of time in culture may be defined as from 30 minutes to 1 hour, from 1 to 6 hours, from 6-12 hours, from 12-24 hours, from 1-7 days, from 7-30 days, or from 1-6 months.

Prior to culturing the non-pluripotent cell in the presence of one or more epigenetic altering agents, the non-pluripotent cell may be cultured in one or more agents designed to maintain the cell actively in mitosis, for all or part of the time that the cell is maintained in culture. Immediately prior to culture in the presence of one or more epigenetic altering agents the cell may be exposed to a treatment designed to drive the cell into a particular stage of the cell cycle or to arrest the cell at a particular location in the cell cycle, such as the S, G₁, M, or G₂ phases. Alternatively, the cell may be induced to exit the cell cycle and enter G₀. Cells in G0 may be obtained directly upon isolation from the mammal, or may be obtained from cells that were initially cycling in culture and where then induced to exit the cell cycle by, for example, removal of serum and mitogen factors. In other embodiments, cells may be derived from a mammal, expanded in culture as described above, and then induced to enter a particular stage of the cell cycle and stop, such as G₀. The cells may then be maintained in culture further prior to exposure to the epigenetic altering agents, such as for about 5 days.

Epigenetic altering agents useful in the invention include, by way of example, DNA-methylation inhibitors and histone-deacetylase inhibitors. By way of example, DNA-methylation inhibitors include nucleoside analogues and non-nucleoside analogues. Exemplary nucleoside analogs include 5-Azacytidine (which may be used at a concentration of 100 nM to 10 μM), 5-Aza-2′-deoxycytidine (which may be used at a concentration of 100 nM to 10 μM), 5-Fluoro-2′-deoxycytidine (which may be used at a concentration of 100 nM to 10 μM), 5,6-Dihydro-5-azacytidine (which may be used at a concentration of 100 nM to 10 μM), and Zebularine (which may be used at a concentration of 1 μM to 10 mM). Exemplary non-nucleoside analogues include Hydralazine (which may be used at a concentration of 100 nM to 10 μM), Procainamide (which may be used at a concentration of 1000 nM to 10 μM), EGCG (which may be used at a concentration of 100 nM to 10 μM), Psammaplin A (which may be used at a concentration of 100 nM to 10 μM), MG98 (which may be used at a concentration of 100 nM to 10 μM), and RG108 (which may be used at a concentration of 100 nM to 10 μM).

Exemplary histone-deacetylase inhibitors include short chain fatty acids, hydroxamic acids, Cyclic tetrapeptides and benzamides, and Benzamides. Exemplary short chain fatty acids include Butyrate (which may be used at a concentration of 1 μM to 10 mM) and Valproic acid (which may be used at a concentration of 1 μM to 10 mM). Exemplary hydroxamic acids include m-Carboxy cinnamic acid bishydroxamic acid (CBHA) (which may be used at a concentration of 100 nM to 10 μM), Oxamflatin (which may be used at a concentration of 100 nM to 10 μM), PDX 101 (which may be used at a concentration of 100 nM to 10 μM), Pyroxamide (which may be used at a concentration of 1 nM to 10 μM), Scriptaid (which may be used at a concentration of 100 nM to 10 μM), Suberoylanilide hydroxamic acid (SAHA) (which may be used at a concentration of 100 nM to 10 μM), Trichostatin A (TSA) (which may be used at a concentration of 1 nM to 10 μM), LBH589 (which may be used at a concentration of 1 nM to 10 μM), and NVP-LAQ824 (which may be used at a concentration of 1 nM to 10 μM). Exemplary cyclic tetrapeptides and benzamides include Apicidin (which may be used at a concentration of 1 nM to 10 μM), Depsipeptide (which may be used at a concentration of 100 nM to 10 μM), TPX-HA analogue (CHAP) (which may be used at a concentration of 1 nM to 10 μM), and Trapoxin (which may be used at a concentration of 1 nM to 10 μM). Exemplary Benzamides include CI-994 (N-acetyldinaline) (which may be used at a concentration of 100 nM to 10 μM) and MS-275 (which may be used at a concentration of 100 nM to 10 μM).

Non-pluripotent cells may be cultured in the in the presence of one or more epigenetic altering agents for 1 to 7 days, or 1 to 2 days, or 2 to 4 days, or 4 to 7 days, or for longer than 7 days, for example. In some embodiments a single epigenetic altering agent is used, while in others multiple agents are used, either concurrently or sequentially. The time periods for culturing given just above may apply to the total time in all epigenetic altering agents or, in embodiments in which the agents are used sequentially, may apply to the time in each agent. In some embodiments a single DNA-methylation inhibitor and a single histone-deacetylase inhibitor are used together, either concurrently or sequentially, while in others multiple DNA-methylation inhibitors are used and/or mulitple histone-deacetylase inhibitors are used.

In some embodiments of the methods of the invention a pluripotent stem cell that expresses one or more mRNA or protein pluripotent stem cell marker that was not expressed in the lineage-restricted stem cell following isolation but before culture in the presence of the epigenetic altering agent is detected. Detection of the mRNA or protein marker may be by any method known in the art. In some embodiments, nucleic acids and/or proteins will be isolated from the cells and then analyzed. Prior to analysis the nucleic acids and/or proteins may be attached to a hybridization support, which may be any substrate that a nucleic acid, polypeptide, or antibody may be attached to for use in an assay comprising a hybridization step. A hybridization support can be porous or solid, planar or non-planar, unitary or distributed. The bond between the nucleic acid or polypeptide and the substrate can be covalent or non-covalent.

Hybridization supports include, but are not limited to, a membrane, such as nitrocellulose, nylon, positively-charged derivatized nylon; a solid substrate such as glass, amorphous silicon, crystalline silicon, plastics (including e.g., polymethylacrylic, polyethylene, polypropylene, polyacrylate, polymethylmethacrylate, polyvinylchloride, polytetrafluoroethylene, polystyrene, polycarbonate, polyacetal, polysulfone, cellulose acetate, or mixtures thereof).

Nucleic acids, polypeptides, and antibodies can be attached covalently a surface of the hybridization support or applied to a derivatized surface in a chaotropic agent that facilitates denaturation and adherence, e.g., by noncovalent interactions, or some combination thereof.

In an embodiment, a hybridization support comprises multiple nucleic acids or polypeptides of attached to a single support, such as a single piece of nitrocellulose membrane or a single glass slide, in an array format, each nucleic acid having a unique physical location on the hybridization support. Such arrays differ mainly by their size, the material of the support and, optionally, the number of nucleic acids that are attached thereto.

In one embodiment, mRNA is isolated from pluripotent stem cells and the hybridized to a solid support containing 1, 1-5, 5-10, 10-20, 20-50, 50-100, 100-200, 200-1000, or 1000-10,000, or 10,000 to 50,000 or more probes. The mRNA may be analyzed directly or further manipulated, such as by fragmentation or by synthesis of cDNA.

In embodiments assaying for the presence of one or more protein marker antibodies may be used, including one or more polyclonal antibodies, monoclonal antibodies, antibody compositions, antibodies having mono- or poly-specificity, humanized antibodies, single-chain antibodies, chimeric antibodies, CDR-grafted antibodies, antibody fragments such as Fab, F(ab′)2, Fv, and other antibody fragments which retain the antigen binding function of the parent antibody. The antibody may be labeled directly, such as covalently, or it may be used in an assay in which a second antibody or binding agent is used to detect the presence of the antibody and, thus, the protein marker.

In other embodiments, the mRNA or protein marker is detected in or on the cell without lysing the cell. Examples of such methods include FACS analysis using antibodies or fluorescently labeled nucleic acids.

Examples of diseases, disorders, or conditions that may be treated or prevented using the methods of the invention include neurological, endocrine, structural, skeletal, vascular, urinary, digestive, integumentary, blood, immune, auto-immune, inflammatory, endocrine, kidney, bladder, cardiovascular, cancer, circulatory, digestive, hematopoeitic, and muscular diseases, disorders, and conditions. In addition, pluripotent stem cells or reprogrammed cells may be used for reconstructive applications, such as for repairing or replacing tissues or organs.

Examples of medical applications for pluripotent stem cells or reprogrammed cells include the administration of neuronal cells to an appropriate area in the human nervous system to treat, prevent, or stabilize a neurological disease such as Alzheimer's disease, Parkinson's disease, Huntington's disease, or ALS; or a spinal cord injury. In particular, degenerating or injured neuronal cells may be replaced by the corresponding cells from a mammal, derived directly or indirectly from pluripotent stem cells or reprogrammed cells. This transplantation method may also be used to treat, prevent, or stabilize autoimmune diseases including, but not limited to, insulin dependent diabetes mellitus, rheumatoid arthritis, pemphigus vulgaris, multiple sclerosis, and myasthenia gravis. In these procedures, the cells that are attacked by the recipient's own immune system may be replaced by transplanted cells. In particular, insulin-producing cells may be administered to the mammal for the treatment or prevention of diabetes, or oligodendroglial precursor cells may be transplanted for the treatment or prevention of multiple sclerosis. For the treatment or prevention of endocrine conditions, reprogrammed cells that produce a hormone, such as a growth factor, thyroid hormone, thyroid-stimulating hormone, parathyroid hormone, steroid, serotonin, epinephrine, or norepinephrine may be administered to a mammal. Additionally, reprogrammed epithelial cells may be administered to repair damage to the lining of a body cavity or organ, such as a lung, gut, exocrine gland, or urogenital tract. It is also contemplated that reprogrammed cells may be administered to a mammal to treat damage or deficiency of cells in an organ such as the bladder, brain, esophagus, fallopian tube, heart, intestines, gallbladder, kidney, liver, lung, ovaries, pancreas, prostate, spinal cord, spleen, stomach, testes, thymus, thyroid, trachea, ureter, urethra, or uterus.

Pluripotent and reprogrammed cells may also be combined with a matrix to form a tissue or organ in vitro or in vivo that may be used to repair or replace a tissue or organ in a recipient mammal. For example, pluripotent and reprogrammed cells may be cultured in vitro in the presence of a matrix to produce a tissue or organ of the urogenital system, such as the bladder, clitoris, corpus cavermosum, kidney, testis, ureter, uretal valve, or urethra, which may then be transplanted into a mammal (Atala, Curr. Opin. Urol. 9(6):517-526, 1999). In another transplant application, synthetic blood vessels are formed in vitro by culturing pluripotent and reprogrammed cells in the presence of an appropriate matrix, and then the vessels are transplanted into a mammal for the treatment or prevention of a cardiovascular or circulatory condition. For the generation of donor cartilage or bone tissue, pluripotent and reprogrammed cells such as chondrocytes or osteocytes are cultured in vitro in the presence of a matrix under conditions that allow the formation of cartilage or bone, and then the matrix containing the donor tissue is administered to a mammal. Alternatively, a mixture of the cells and a matrix may be administered to a mammal for the formation of the desired tissue in vivo. The cells may be attached to the surface of the matrix or encapsulated by the matrix. Examples of matrices that may be used for the formation of donor tissues or organs include collagen matrices, carbon fibers, polyvinyl alcohol sponges, acrylateamide sponges, fibrin-thrombin gels, hyaluronic acid-based polymers, and synthetic polymer matrices containing polyanhydride, polyorthoester, polyglycolic acid, or a combination thereof (see, for example, U.S. Pat. Nos. 4,846,835; 4,642,120; 5,786,217; and 5,041,138).

A reduction in DNA methylation may be achieved by using an antibody against 5-methyl cytosine which binds to methylated DNA. Levels of total cellular methylation can be quantified by flow cytometry through measuring the fluorescent levels of cells after incubation in anti 5-methyl cytosine primary antibody and fluorescent-conjugated secondary antibodies. Methylation levels of specific genes can be measured by using this antibody in a chromatin immunoprecipitation (ChIP) procedure which can then be hybridized to a DNA microarray, for example. Levels of histone acetylation can be measured globally using flow cytometry as above or by Western blot except, in both cases, anti-acetylated histone H3 antibodies are used instead of anti 5-methyl cytosine antibodies. These antibodies can also be used for ChIP as above to determine levels of acetylation on a gene by gene basis.

For further elaboration of general techniques useful in the practice of this invention, the practitioner can refer to standard textbooks and reviews in cell and tissue culture, embryology, and molecular biology. General techniques in cell culture and media collection are outlined in Large Scale Mammalian Cell Culture (Hu et at., Curr. Opin. Biotechnol. 8:148, 1997); Serum-free Media (K. Kitano, Biotechnology 17:73, 1991); Large Scale Mammalian Cell Culture (Curr. Opin. Biotechnol. 2:375, 1991); and Suspension Culture of Mammalian Cells (Birch et al., Bioprocess Technol. 19:251, 1990). Textbooks on the subject include General Techniques in Cell Culture (Harrison & Rae, Cambridge, 1997); Animal Cell Culture Methods (Barnes & Mather, eds., Academic Press, 1998); Culture of Animal Cells (I. Freshney, 4th ed., John Wiley & Sons, 2000); Guidebook to the Extracellular Matrix, Anchor, and Adhesion Proteins (Kreis & Vale, eds., Oxford, 1999); Handbook of Cellular Manufacturing Systems (S. A. Irani, ed., John Wiley & Sons, 1999).

The properties, culture, and differentiation of embryonic stem cells are described in Teratocarcinomas and embryonic stem cells: A practical approach (E. J. Robertson, ed., IRL Press Ltd. 1987); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., al., 1993). Differentiation of stem cells is reviewed in Robertson, Meth. Cell Biol. 75:173, 1997; and Pedersen, Reprod. Fertil. Dev. 10:31, 1998. References that further describe the culturing of particular cell types are listed further on in the disclosure.

General biochemical techniques are described in Short Protocols in Molecular Biology (Ausubel et al., eds., 4th ed. 1999). Methods of protein chemistry are described generally in Protein Methods (Bollag et al., 1996); Guide to Protein Purification (Deutscher et al., eds., Methods Enzymol. vol. 182, Academic Press, 1997); Protein Analysis and Purification (I. M. Rosenberg, Springer Verlag, 1996).

Additional objects and advantages of the invention not set forth in the description will be obvious from the description, or may be learned by practice of the invention. The objects and advantages of the invention will be realized and attained by means of the elements and combinations particularly pointed out in the appended claims. Moreover, advantages described in the body of the specification, if not included in the claims, are not per se limitations to the claimed invention.

It is to be understood that the description provided herein is exemplary and explanatory only and is not restrictive of the invention, as claimed. Moreover, it must be understood that the invention is not limited to the particular embodiments described, as such may, of course, vary. Further, the terminology used to describe particular embodiments is not intended to be limiting, since the scope of the present invention will be limited only by its claims. The claims do not encompass embodiments in the public domain.

With respect to ranges of values, the invention encompasses each intervening value between the upper and lower limits of the range. Further, the invention encompasses any other stated intervening values. Moreover, the invention also encompasses ranges excluding either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test the invention.

The specification is most thoroughly understood in light of the references cited herein.

It must be noted that, as used herein and in the appended claims, the singular forms “a,” “or,” and “the” include plural referents unless the context clearly dictates otherwise. Thus, for example, reference to “a pluripotent stem cell” includes a plurality of such stem cells and reference to “the epigenetic altering agent” includes reference to one or more agents and equivalents thereof known to those skilled in the art, and so forth.

Further, all numbers used in the specification and claims are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

### EXAMPLES

The following examples are provided in order to demonstrate and further illustrate certain preferred embodiments and aspects of the present invention and are not to be construed as limiting the scope thereof.

### Example 1

In this example chromatin modifying agents are used to increase numbers of adipose tissue stem cells with high aldehyde dehydrogenase activity (ALDH). ALDH has been identified as a unique marker of stem cells displaying high differentiation and self-renewal potential (see review article: Cai et al. In search of “stemness”. Experimental Hematology, 32, 585-598). Several types of somatic stem cells, including hematopoietic (HSC), cord blood (CB) and neural stem cells (NSC) display, preferentially, high levels of ALDH. In this example chromatin modifying agents are used to bolster the number and intensity of ALDH positive cells within a population of adipose tissue stem cells.

Adipose tissue derived stem cells (ASCs) with a CD34+CD105+CD31−CD45− cell surface phenotype were freshly isolated from human lipoaspirate using the procedure described in Boquest et al., “Isolation and transcription profiling of purified uncultured human stromal stem cells: alteration of gene expression after in vitro cell culture,” Molecular Biology of the Cell, 16, 113 1-1141, 2005). 0.2×106 freshly isolated ASCs were pelleted by centrifugation (300 g for 10 minutes) and resuspended in cell culture media (DMEM/F12) containing 20% fetal calf serum and chromatin modifying agents at the following concentrations and combinations: (1) control, no trichostatin A (TSA) or 5-azacytidine (5-azaC); (2) TSA (0.5 μM); (3) TSA (1 μM); (4) TSA (0.5 μM) and 5-azaC (3 μM); (5) TSA (1 μM)+5-azaC (3 μM). Cells were incubated at 37° C. in an atmosphere of 5% CO₂, 20% O₂, in air for 48 hours in 25 cm² cell culture flasks. To expand cells, treatment media was then removed from adhered cells and replaced with culture media (DMEM/F12) containing 20% fetal calf serum but without chromatin modifying agents (i.e., neither TSA nor 5-azaC was present). After 1 week of culture, cells were then sub-cultured using standard procedures, followed by further sub-culturing every 3-4 days until passage 5. At that point, the cells have undergone approximately 15 population doublings. The cells (10⁶ per treatment) were then stained for ALDH activity using the ALDEFLUOR assay kit and instructions provided (StemCo Biomedical, Inc. North Carolina, USA). Cells brightly positive for ALDH (ALDH+) were then quantified using flow cytometry according to the manufacturers specifications (StemCo Biomedical). The intensity of fluorescence in ALDH+ of each treatment was also compared with control, untreated cells.

Percentages of ALDH+ ASCs after treatment with chromatin modifying agents were calculated. Results from an average of 2 replicates were used for each donor. Percentages of ALDH+ cells are significantly higher when ASCs were initially treated with a combination of TSA and 5-azaC. The greatest response is apparent when 0.5 μM TSA and 3 μM 5-azaC was used, as evidenced by a 3-fold increase in ALDH+ cells compared with untreated cells. Furthermore, the same treatment results in a population of ALDH+ ASCs with elevated levels of the ALDH enzyme, as reflected by higher fluorescent intensity. Higher percentages of ALDH+ cells with elevated levels of ALDH as a result of treatment with a combination of TSA and 5-azaC suggests that such treatment induces greater differentiation and self renewal capacity in ASCs.

### Example 2

In this example chromatin modifying agents were used to over express genes associated with pluripotency in adipose tissue stem cells. Over expression of genes including OCT-4, NANOG, Telomerase, SOX-2, and REX-1 has been specifically associated with pluripotential cell types including embryonic stem cells, embryonal carcinoma cells, and the inner cell mass of pre-implantation embryos. In this example chromatin modifying agents were used to induce expression of these genes in adipose tissue stem cells (ASCs).

Adipose tissue derived stem cells (ASCs) with a CD34+CD105+CD31−CD45− cell surface phenotype were freshly isolated from human lipoaspirate using the procedure described in Boquest et al., “Isolation and transcription profiling of purified uncultured human stromal stem cells: alteration of gene expression after in vitro cell culture,” Molecular Biology of the Cell, 16, 113 1-1141, 2005). 0.2×106 freshly isolated ASCs are pelleted by centrifugation (300 g for 10 minutes) and resuspended in cell culture media (DMEM/F12) containing 20% fetal calf serum and chromatin modifying agents at the following concentrations and combinations: (1) control, no trichostatin A (TSA) or 5-azacytidine (5-azaC); or (2) TSA (.1 μM)+5-azaC (3 μM). Cells were incubated at 37° C. in an atmosphere of 5% CO₂, 20% O₂, in air for 48 hours in 25 cm² cell culture flasks. To expand cells, treatment media was removed from adhered cells and replaced with culture media (DMEM/F12) containing 20% fetal calf serum but without chromatin modifying agents. After 1 week of culture, cells were then sub-cultured using standard procedures, followed by further sub-culturing every 3-4 days until passage 3. At that point, the cells had undergone approximately 10 population doublings. The cells (0.5×10⁶ per treatment) were then assayed for the presence of mRNA products of the following genes using quantitative real time RT-PCR: OCT-4, NANOG, Telomerase, SOX-2, REX-1.

Fold differences in gene expression were seen when comparing treated cells, ASCs treated with 0.1 μM TSA+3 μM 5-azaC for 24 hours, to untreated cells, such that treatment of ASCs with a combination of TSA and 5-azaC leads to over expression of genes associated with pluripotency. The cells were analyzed at P3, representing about 10 population doublings post treatment.

### Example 3

This example describes a method that can be used to produce functional pancreatic β-cells from adipose tissue stem cells (ASCs) using chromatin modifying agents to overcome juvenile diabetes. Adipose tissue derived stem cells (ASCs) with a CD34+CD105+CD31−CD45− cell surface phenotype will be freshly isolated from human lipoaspirate of a patient with juvenile diabetes using the procedure described in Boquest et al., “Isolation and transcription profiling of purified uncultured human stromal stem cells: alteration of gene expression after in vitro cell culture,” Molecular Biology of the Cell, 16, 1131-1141, 2005). Freshly isolated ASCs which are lineage restricted in their ability to only form tissues within the mesoderm germ layer (such as bone, fat, muscle) are then reprogrammed using chromatin modifying agents to form cells having a higher state of plasticity (pluripotent), as evidenced by their new ability to also form functional tissues of the other two germ layers, namely endoderm (such as pancreatic cells, liver cells, ect.) and ectoderm (cells of central nervous system, skin cells, ect.). Specifically, 10⁶ freshly isolated ASCs are pelleted by centrifugation (300 g for 10 minutes) and resuspended in cell culture media (DMEM/F12) containing 20% fetal calf serum and chromatin modifying agents: trichostatin A (1 μM) and 5-azacytidine (3 μM), as well as 10 ng/ml bFGF (a factor that promotes stem cell self renewal and therefore symmetrical cell division) and incubated at 37° C. in an atmosphere of 5% CO₂, 5% O₂, 90% N for 48 hours in a 25 cm² cell culture flask. To expand cells, media is then removed from adhered cells and replaced with the appropriate culture media (most likely media used to culture embryonic stem cells), but without chromatin modifying agents. Cells are then sub-cultured using standard procedures every 3-4 days until the required number of cells is reached. The resultant cell population resembles that of embryonic stem (ES) cells: (1) they now over express key genes related to pluripotent ES cells including OCT-4, NANOG, TERT and SOX-2; (2) their morphology now resembles that of ES cells, being tightly packed-together with scant cytoplasm and large nucleus; and (3) If placed into an early embryo, these cells would form functional tissues of all three germ layers.

A sub population of these reprogrammed, pluripotent cells may then be frozen in liquid nitrogen for future use. If the patient in the future requires additional tissue replacement, for example neuronal tissue after spinal cord injury, they can be thawed, expanded in culture and differentiated into functional neurons (ectoderm).

The population of cells remaining in culture is then differentiated into pancreatic tissue using protocols for the differentiation of ES cells towards the pancreatic β-cell pathway. Pluripotent genes are down regulated and genes relating to pancreatic tissues are unregulated (such as PDX-1). The resulting differentiated, or partially differentiated pancreatic β-cells are then transplanted back into the patient resulting in the normalization of circulating blood glucose levels.

### Example 4

This example describes a method that can be used to produce cloned pigs from parthenotes aggregated with porcine hematopoetic stem cells (HSCs) reprogrammed using chromatin modifying agents. Porcine bone marrow is collected from the pig to be cloned. CD34+ HSCs are separated from the bone marrow aspirates using magnetic beads conjugated with anti CD34 porcine antibody. 10⁶ freshly isolated HSCs are pelleted by centrifugation (300 g for 10 minutes) and resuspended in cell culture media (DMEM/F12) containing 20% fetal calf serum and chromatin modifying agents: trichostatin A (1 μM) and 5-azacytidine (3 μM), as well as 10 ng/ml bFGF (a factor that promotes stem cell self renewal and therefore symmetrical cell division) and incubated at 37° C. in an atmosphere of 5% CO₂, 5% O₂, 90% N for 48 hours in a 25 cm² cell culture flask. Treatment media is removed from cells and replaced with appropriate media, such as embryonic cell culture media, but without trichostatin A and 5-azacytidine, for further expansion. The cells and their progeny resemble porcine ES cells.

To make cloned pigs from these cells, blastocysts produced by activation of in vitro matured sow oocytes are used as surrogate embryos. One hundred reprogrammed HSCs are aggregated with inner cell mass cells of a day 6 porcine parthenote. At least 20 aggregated blastocyts are subsequently transferred to a uterus of a synchronized, day 6 gilt leading to the birth of normal, cloned piglets just under 4 months later.

### Example 5

This example describes a method that allows for the efficient production of embryonic stem cell (ESC) lines from cloned human embryos using chromatin modifying agents for cell therapy applications. A fibroblast cell line is created from a patient with congenital heart disease. Using standard methods of somatic cell nuclear transfer, a fibroblast cell from the patient is fused with an enucleated human oocyte and subsequently activated to form a 1-cell cloned embryo. The cloned embryo is placed, immediately after activation, in standard human embryo culture medium containing 50 nm Trichostatin A for 10 hours. The embryo is then washed and cultured in human embryo culture medium without Trichostatin A for a further 6 days. Embryonic stem cells are then harvested from the embryo after reaching the blastocyst stage and cultured using established procedures to form an ESC line. Cells from this ESC line are then differentiated into beating heart tissue using established procedures. The functional heart tissue is subsequently transferred back to the heart of the patient in order to ameliorate the congenital heart condition.

### Example 6

This example describes a method that allows for the efficient production of cloned pigs for agricultural purposes using chromatin modifying agents. A fibroblast cell line is created from the boar to be cloned. Using standard methods of somatic cell nuclear transfer, a fibroblast cell from the boar is fused with an enucleated porcine oocyte and subsequently activated to form a 1-cell cloned embryo. The cloned embryo is placed, immediately after activation, in standard porcine embryo culture medium containing 50 nm Trichostatin A for 10 hours. The embryo is then washed and immediately transferred to a synchronized recipient sow for gestation and subsequent birth of a normal cloned piglet.

### Example 7

This example describes the use of chromatin modifying agents in improving the outcome of human in vitro fertilization (IVF). An oocyte from the sub-fertile female is incubated with sperm to produce a zygote using standard IVF methods. Immediately after fertilization, the zygote is incubated in standard human embryo culture medium containing 50 nm Trichostatin A for 10 hours. The zygote is then washed and cultured in human embryo culture medium without Trichostatin A for a further two to five days before being transferred back to the sub-fertile female or frozen for transfer at a later date.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method of making a pluripotent stem cell from a cell that is not pluripotent, comprising:
isolating a non-pluripotent cell from a tissue of a mammal; and
culturing the non-pluripotent cell in the presence of one or more epigenetic altering agents, under conditions that allow formation of a pluripotent stem cell, to thereby provide the pluripotent stem cell.

2. The method of claim 1, wherein the isolated non-pluripotent cell is in a G₀ state.

3. The method of claim 1, wherein the isolated non-pluripotent cell is terminally differentiated.

4. A method of making a pluripotent stem cell from a lineage-restricted stem cell, comprising:
isolating a lineage-restricted stem cell from a tissue of a mammal; and
culturing the lineage-restricted stem cell in the presence of one or more epigenetic altering agents, under conditions that allow formation of a pluripotent stem cell, to thereby provide the pluripotent stem cell.

5. The method of claim 4, wherein the isolated lineage-restricted stem cell is in a G₀ state.

6. The method of claim 4, wherein the lineage-restricted stem cell is a somatic stem cell.

7. The method of claim 6, wherein the somatic stem cell is adipose tissue-derived.

8. The method of claim 4, wherein the lineage-restricted stem cell is a stromal stem cell.

9. The method of claim 4, wherein the lineage-restricted stem cell is a CD45−, CD34+, CD 105+ cell.

10. The method of claim 9, wherein the lineage-restricted stem cell is also CD31−.

11. The method of claim 4, wherein the lineage-restricted stem cell is obtained from a tissue selected from adipose, bone marrow, blood, brain, muscle, skin, liver, and pancreas.

12. The method of claim 4, wherein the lineage-restricted stem cell is selected from an adipose tissue stromal stem cell, bone marrow stromal stem cell, hematopoietic stem cell, hair follicle stem cell, neural stem cell, muscle stem cell, cord blood stem cell, skin stem cell, liver stem cell, and pancreatic stem cell.

13. The method of claim 4, wherein the one or more epigenetic altering agents is a histone deacetylase inhibitor.

14. The method of claim 13, wherein the histone deacetylase inhibitor is trichostatin A.

15. The method of claim 4, wherein the one or more epigenetic altering agents is a DNA methyltransferase inhibitor.

16. The method of claim 15, wherein the DNA methyltransferase inhibitor is 5-azacytidine.

17. The method of claim 4, wherein the one or more epigenetic altering agents comprises a histone deacetylase inhibitor and a DNA methyltransferase inhibitor.

18. The method of claim 17, wherein the histone deacetylase inhibitor is trichostatin A and the DNA methyltransferase inhibitor is 5-azacytidine.

19. The method of claim 4, wherein one or more chromatin marker of an increased developmental potential is increased in the pluripotent stem cell relative to the lineage-restricted stem cell following isolation but before culture in the presence of the epigenetic altering agent.

20. The method of claim 19, wherein the chromatin modification comprises a reduction in DNA methylation.

21. The method of claim 19, wherein the chromatin modification comprises an increase in histone acetylation.

22. The method of claim 19, wherein the chromatin modification comprises a reduction in DNA methylation and an increase in histone acetylation.

23. The method of claim 4, wherein the pluripotent stem cell expresses one or more mRNA or protein pluripotent stem cell marker that was not expressed in the lineage-restricted stem cell following isolation but before culture in the presence of the epigenetic altering agent.

24. The method of claim 23, wherein the pluripotent stem cell expresses 1, 1-5, 5-10, 10-20, 20-50, 50-100, 100-200, 200-1000, or over 1000 mRNA or protein pluripotent stem cell markers that were not expressed in the lineage-restricted stem cell following isolation but before treatment with the epigenetic altering agent.

25. The method of claim 23, wherein the one or more mRNA or protein marker is one or more of OCT-4, TERT, NANOG, SSEA-1, TRA-1-60, TRA-1-81, AC133, CD9, FLT3, C-KIT, REX-1, STELLA, SOX-2, UTF1, OXT2, LEFTY-1, FOXD3, DNMT3A, DNMT3B, and FGF2.

26. The method of claim 4, wherein the mammal is a human, cow, sheep, big-horn sheep, goat, buffalo, antelope, oxen, horse, donkey, mule, deer, elk, caribou, water buffalo, camel, llama, alpaca, rabbit, pig, mouse, rat, guinea pig, hamster, dog, cat, or primate, such as a monkey.

27. The method of claim 26, wherein the mammal is a mouse.

28. The method of claim 26, wherein the mammal is a human.

29. The method of claim 4, wherein the isolated lineage-restricted stem cell is a progeny of at least one cell division that occurred in vitro, prior to culturing in the presence of one or more epigenetic altering agents

30. A pluripotent stem cell made by the method of claim 4.

31. A method for making a pluripotent stem cell from a lineage-restricted stem cell, comprising:
isolating a lineage-restricted stem cell from a tissue of a mammal, wherein the lineage-restricted stem cell expresses one or more mRNA or protein markers that define its lineage restriction; and
exposing the lineage-restricted stem cell to one or more epigenetic altering agents, to thereby provide the pluripotent stem cell;
wherein the pluripotent stem cell expresses one or more mRNA or protein pluripotent stem cell marker that was not expressed in the lineage-restricted stem cell following isolation but before treatment with the epigenetic altering agent; and
wherein the pluripotent stem cell does not express one or more mRNA or protein marker that define the lineage restriction of the lineage-restricted stem cell from which it was made.

32. The method of claim 31, wherein the pluripotent stem cell is a progeny of the lineage-restricted stem cell.

33. A method of providing a reprogrammed cell, comprising:
making a pluripotent stem cell from a lineage-restricted stem cell, by a method comprising:
isolating a lineage-restricted stem cell from a tissue of a mammal;
culturing the lineage-restricted stem cell in the presence of one or more epigenetic altering agents, under conditions that allow formation of a pluripotent stem cell, to thereby provide the pluripotent stem cell; and
culturing the pluripotent stem cell in one or more reprogramming agents, under conditions that allow formation of a reprogrammed cell, to thereby provide the reprogrammed cell.

34. The method of claim 33, wherein the reprogrammed cell expresses one or more mRNA or protein reprogramming marker that was not expressed in the lineage-restricted stem cell.

35. The method of claim 34, wherein the reprogrammed cell is a lineage-restricted stem cell.

36. A reprogrammed cell made by the method of claim 33.

37. A method of treating or preventing a disease, disorder, or condition in a mammal, comprising:
isolating a lineage-restricted stem cell from a tissue of a mammal;
culturing the lineage-restricted stem cell in the presence of one or more epigenetic altering agents, under conditions that allow formation of a pluripotent stem cell, to thereby provide the pluripotent stem cell; and
administering the pluripotent stem cell to a mammal in need of cells derived from the pluripotent stem cell.

38. The method of claim 37, wherein the lineage-restricted stem cell is isolated from the mammal that receives the pluripotent stem cell.

39. A method of treating or preventing a disease, disorder, or condition in a mammal, comprising:
making a pluripotent stem cell from a lineage-restricted stem cell, by a method comprising:
isolating a lineage-restricted stem cell from a tissue of a mammal;
culturing the lineage-restricted stem cell in the presence of one or more epigenetic altering agents, under conditions that allow formation of a pluripotent stem cell, to thereby provide the pluripotent stem cell;
culturing the pluripotent stem cell in one or more reprogramming agents, under conditions that allow formation of a reprogrammed cell, to thereby provide the reprogrammed cell; and
administering the reprogrammed cell to a mammal in need of cells of the reprogrammed cell type.

40. The method of claim 39, wherein the lineage-restricted stem cell is isolated from the mammal that receives the reprogrammed cell.

41. A mammal comprising a cell administered by the method of claim 39.

42. A method of inducing or increasing expression of one or more mRNA or protein pluripotent stem cell markers in a cell, comprising:
determining the expression level of the one or more mRNA or protein pluripotent stem cell markers in the cell when the cell is cultured in the absence of one or more epigenetic altering agents;
culturing the cell in the presence of the one or more epigenetic altering agents;
determining the expression level of the one or more mRNA or protein pluripotent stem cell markers in the cell when the cell is cultured in the presence of the one or more epigenetic altering agents; and
comparing the expression level of the one or more mRNA or protein pluripotent stem cell markers when the cell is cultured in the presence of the one or more epigenetic altering agents, with the expression level of the one or more mRNA or protein pluripotent stem cell markers when the cell is cultured in the absence of the one or more epigenetic altering agents, and determining that the expression level of the one or more mRNA or protein pluripotent stem cell markers is induced or increased when the cell is cultured in the presence of the one or more epigenetic altering agents.

43. The method of claim 42, wherein the one or more epigenetic altering agents is a histone deacetylase inhibitor.

44. The method of claim 43, wherein the histone deacetylase inhibitor is trichostatin A.

45. The method of claim 42, wherein the one or more epigenetic altering agents is a DNA methyltransferase inhibitor.

46. The method of claim 45, wherein the DNA methyltransferase inhibitor is 5-azacytidine.

47. The method of claim 42, wherein the one or more epigenetic altering agents comprises a histone deacetylase inhibitor and a DNA methyltransferase inhibitor.

48. The method of claim 47, wherein the histone deacetylase inhibitor is trichostatin A and the DNA methyltransferase inhibitor is 5-azacytidine.

49. The method of claim 42, wherein the expression level of 2-5, 5-10, 10-20, 20-50, 50-100, 100-200, 200-1000, or over 1000 mRNA or protein pluripotent stem cell markers is induced or increased by culturing the cell in the presence of the one or more epigenetic altering agents.

50. The method of claim 42, wherein the one or more mRNA or protein marker selected from OCT-4, TERT, NANOG, SSEA-1, SSEA-4, TRA-1-60, TRA-1-81, AC133, CD9, FLT3, C-KIT, REX-1, STELLA, SOX-2, UTF1, OXT2, LEFTY-1, FOXD3, DNMT3A, DNMT3B, and FGF2.

51. The method of claim 42, wherein the cell is a lineage-restricted stem cell.

52. The method of claim 51, wherein the lineage-restricted stem cell is a somatic stem cell.

53. The method of claim 52, wherein the somatic stem cell is adipose tissue-derived.

54. The method of claim 51, wherein the lineage-restricted stem cell is a stromal stem cell.

55. The method of claim 51, wherein the lineage-restricted stem cell is a CD45−, CD34+, CD 105+ cell.

56. The method of claim 55, wherein the lineage-restricted stem cell is also CD31−.

57. A method of making a cloned mammalian cell, comprising:
isolating a non-pluripotent cell from a tissue of a mammal;
culturing the cell in the presence of one or more epigenetic altering agents, under conditions that allow the chromatin of the cell to be modified such that the developmental potential of the cell is increased;
transferring the nucleus of the cell with an increased developmental potential into an enucleated recipient cell; and
allowing the recipient cell to undergo one or more cell divisions to provide the cloned mammalian cell.

58. The method of claim 57, wherein the cloned mammalian cell is a pluripotent stem cell.

59. A method of treating or preventing a disease, disorder, or condition in a mammal, comprising:
making a pluripotent stem cell by the method of claim 58; and
administering the pluripotent stem cell to a mammal in need thereof.

60. The method of claim 58, further comprising culturing the pluripotent stem cell in one or more reprogramming agents, under conditions that allow formation of a reprogrammed cell, to thereby provide a reprogrammed cell.

61. The method of claim 60, wherein the reprogrammed cell is a lineage-restricted stem cell.

62. A method of treating or preventing a disease, disorder, or condition in a mammal, comprising:
making a cloned cell by the method of claim 61; and
administering the cloned cell to a mammal in need of a reprogrammed cell.

63. A method of making a cloned mammal, comprising transferring a cell according to claim 57 into a recipient blastula;
transferring the blastula into a recipient mother; and
allowing the blastula to develop to term to thereby provide the cloned mammal.

64. A method of making a cloned cell, comprising isolating a cloned cell from the cloned mammal of claim 63.

65. A method of treating or preventing a disease, disorder, or condition in a mammal, comprising:
making a cloned cell by the method of claim 64; and
administering the cloned cell to a mammal in need thereof.

66. A method of making a cloned mammalian embryo, comprising transferring a cell according to claim 57 into a recipient blastula;
transferring the blastula into a recipient mother; and
allowing the blastula to develop to an embryonic stage to provide the cloned mammalian embryo.

67. A method of making a cloned cell, comprising isolating a cloned cell from the cloned mammalian embryo of claim 66.

68. A method of treating or preventing a disease, disorder, or condition in a mammal, comprising:
making a cloned cell by the method of claim 67; and
administering the cloned cell to a mammal in need thereof.
